(19) United States
(12) Patent Application Publication
(10) Pub. No.: US 2005/0187457 A1
(43) Pub. Date: Aug. 25, 2005

(54) Mr imaging method

(76) Inventor: Hans Gerard Jenniskens, Eindhoven (NL)

Correspondence Address: Thomas M Lundin;Philips Intellectual Property & Standards, 595 Miner Road, Cleveland, OH, 44143, US

(21) Appl. No.: 10/516,138

(22) PCT Filed: May 27, 2003
(86) PCT No.: PCT/IB03/02035

(30) Foreign Application Priority Data
May 30, 2002 (EP) 02077127.5

(51) Int. Cl. A61B 5/05

(52) U.S. Cl. 600/410

(57) ABSTRACT

The invention relates to an MR imaging method in which at least one gradient magnetic field which traverses the object (7) to be examined is applied for a given period of time, a co-ordinate system being associated with the object (7) to be examined. In most cases the parameters of the MR method are already determined by the medical task. It is an object of the invention to provide an MR method which offers, without having to sacrifice imaging possibilities existing thus far, a possibility for controlling the direction of the shift (11) of parts of the object to be examined which exhibit deviantly resonating spins. In accordance with the invention the object is achieved by means of an MR imaging method of the kind set forth in which the orientation of at least one field strength gradient (13) of a gradient magnetic field along an axis of the magnet co-ordinate system is provided as a measuring parameter. An advantage of the invention is formed by the enhanced possibilities for imaging.

The invention relates to a method for the MR imaging of an object to be examined by means of an MR apparatus which includes at least one main magnetic field device and at least one gradient magnetic field device for position encoding, in which a control algorithm controls measuring sequences in conformity with predetermined measuring parameters, in which the magnetic moment of the spins is aligned on average in a preferred direction by means of a main magnetic field, in which at least one gradient magnetic field which traverses at least partly the object to be examined and whose field strength exhibits a field strength gradient which is at least partly essentially constant in at least one spatial direction is applied for a given period of time, in which the preferred direction of the magnetic field generated by means of the main magnetic field device defines an axis of a stationary magnetic co-ordinate system, in which at least one further axis of the magnet co-ordinate system is defined by the direction of the field strength gradient of the magnetic field which can be generated by means of a gradient magnetic field device, in which axes of a measuring co-ordinate system are defined each time by the direction of the slices and/or the direction of the phase encoding and/or the reading direction on the object to be examined, and in which a co-ordinate system is associated with the object to be examined.

An MR apparatus comprises essentially a strong magnet which generates a steady main magnetic field and a plurality of gradient coils which generate a respective gradient magnetic field in each of the three spatial directions. Generally speaking, amplifiers are associated with the gradient coils. Parts of the object to be examined are excited by means of an RF transmitter. The MR signal is picked up and amplified by means of a very sensitive RF receiver; during this operation an automatic switch frequently makes the transmission coil also suitable for the reception. Furthermore, an MR apparatus includes several computers for executing the control algorithm, for controlling the apparatus, for reconstructing the MR images, for co-ordinating the various processes, notably during the measurement, for enabling entries via the control console and for filing the acquired images.

In the context of a typical magnetic resonance imaging method the magnetic moment of the protons is aligned in one spatial direction by means of a strong, steady magnetic field of from approximately 1.5 to 3 Tesla Using brief electromagnetic RF pulses, the individual protons are excited to precession and subsequently they are aligned again in conformity with the external strong magnetic field. Notably the excitation and relaxation times as well as the frequencies of the precessional motions are dependent on the tissue and in the context of the measurement they provide, in conjunction with the position encoding of the excitation, information on the situation in space of different tissues. The position encoding utilizes position-dependent frequencies and phases of the precessional excitation and offers, via Fourier transformation of the measured MR signals, information as regards the position of the relevant emission. In order to generate the magnetic field strength gradients desired for position encoding, generally use is made of three different coil systems which extend in three mutually perpendicular spatial directions. The two spatial directions which are oriented perpendicularly to the longitudinal axis of the body are generally referred to as the x direction and the y direction.

Differently resonating spins cause a variety of artifacts in MR imaging. One such artifact is the chemical shift which is due to the fact that protons in fat tissue (hydrogen bound to carbon) and protons in water (hydrogen bound to oxygen) are subject to a magnetic field of different strength due to their different environments. Therefore, they have a resonance frequency which is slightly shifted relative to one another, the difference between their resonance frequencies increasing as the strength of the outer main magnetic field is greater. For example, in the case of spin echo sequences, this phenomenon becomes manifest in the MR images as a shift of the fat tissue towards the structures imaged on the basis of water in the reading direction. After half the echo time, the spin echo sequence applies a 180° pulse which causes, after the full echo time, the rephasing of the dephased spins and hence a strong signal. However, a problem is encountered in that, if an acceptable expenditure on time is to be achieved, the individual measuring values must be acquired at different instants after the excitation pulse or the 180° pulse, so that the undesirable artifacts may arise.

Extraordinarily resonating spins may also be due to inhomogeneities of the main magnetic field. This is the case notably when the tissue to be examined locally exhibits different susceptibilities, for example, due to metal implants or air/tissue interfaces so that the main magnetic field is no longer sufficiently homogeneous. The local variations of the susceptibility cause undesirable local gradients of the main magnetic field. As a result, the frequency of the measured signal is shifted, such a shift being proportional to the local variations of the main magnetic field. Depending on the direction of the gradient of the gradient magnetic field and the gradient caused by the changed susceptibility of the main magnetic field, the causal inhomogeneous part of the object to be examined is represented in the image formed in a compressed or expanded form. Compression of the representation of parts of the image formed is problematic notably because the structure of the regions shown is then usually distorted to such an extent that medical information or a diagnosis can no longer be derived therefrom. Moreover, the compression leads to a local increase of the intensity of the signal; this may give rise to bright spots in the image formed. These effects are highly undesirable already because of the fact that they may give rise to misinterpretation of the image and to incorrect diagnoses.

Spins resonating in a deviant manner also cause problems in respect of the excitation of individual slices, because the deviantly resonating spins can also be excited outside the slice to be excited. Consequently, undesirable changes of contrast may arise in the image formed, because the signal emitted by the tissue containing water in some slices is superposed on the signal from the tissue containing fat from neighboring slices. The demarcation of individual slices to be imaged relative to one another is substantially impeded by such artifacts.

In conventional MR methods the direction of such types of shift can be controlled by a suitable choice of the scan technique, the orientation of the slices and the spatial direction of the phase encoding. Such control of the shift direction by one of these parameters, however, has the drawback that this parameter is no longer available for the further formation of the image. The scan technique in particular is dictated already by the necessary tissue contrast, the scan time and the desired insensitivity to changes of the susceptibility. In most cases the orientation of the slices is already governed by the relevant medical task and cannot be freely chosen either because of so-called fold-over problems. Moreover, the phase encoding direction is already predetermined in most cases in order to avoid fold-over artifacts.

Considering the drawbacks and problems of the present state of the art, it is an object of the invention to provide an MR imaging method whereby the shift of parts of the object to be examined which exhibit deviating resonating spins can be controlled in respect of direction without having to sacrifice any of the known possibilities for forming the MR image.

The object is achieved in accordance with the invention by means of an MR imaging method of the kind set forth in which the orientation of at least one field strength gradient, generated during the measurement, of a gradient magnetic field along an axis of the measuring co-ordinate system is provided as a measuring parameter for the method.

A special advantage of the invention resides in the fact that the operator of the MR apparatus can freely choose the directions of the shift of the parts of the examination volume with deviantly resonating spins, without having to sacrifice other possibilities for imaging. The operator can thus control at will the direction of the shift of fat-containing tissue or the shifts caused by inhomogeneities of the main magnetic field.

In order to ensure that the operator of the MR apparatus need not deal with excessive geometrical aspects, it is advantageous when the orientation of the field strength gradient of the gradient magnetic field is provided in the form of direction indications related to the co-ordinate system of the object to be examined. The operator can thus concentrate better on the medical task.

In an advantageous further version of the invention the control algorithm asks the operator of the MR apparatus for the desired direction of the shift of fat tissue in the image when the desired direction of the field strength gradient is selected on the control console. As a result, the operator of the MR apparatus, usually being entrusted with medical tasks, will not be confronted with an abstract task but will only be requested to answer a comprehensible question. Even though the decision taken by the operator influences not only the so-called “fat shift”, this approach facilitates the procedure considerably.

Another possibility for facilitating the selection of the desired direction of the field strength gradient by the operator at the control console, that is, notably in the case of excitation of individual slices, is given by the fact that the control algorithm requests the operator at the control console to indicate the desired direction of the shift of the slice to be excited.

In order to facilitate the operation of the MR apparatus even further while using the method in accordance with the invention, it is advantageous when, upon selection of the desired direction of the field strength gradient, the control algorithm asks the operator of the MR apparatus for the desired direction of the shift of fat tissue in the image along a predetermined axis of the co-ordinate system of the object to be examined, so that the operator has to choose from two possibilities only. Thus, the operator need not worry about the geometrical circumstances without a mental support, that is, practically without the possibilities of imaging being limited by this choice.

In order to ensure that a logical sequence of electromagnetic pulses and magnetic fields is always obtained during the measurement, it makes sense for the control algorithm to determine the relevant axes for the shift of the fat tissue in the image from the other parameters of the position encoding, and to request from the operator as input the respective desired direction of the shift of fat tissue in the image associated with these axes.

In conformity with a version of the invention, the measuring method is given only the direction of the field strength gradient of a gradient magnetic field device in direction indications related to the co-ordinate system of the object to be examined, that is, each time prior to the measurement. This may be effective for given sequences and may also facilitate the operation of the MR apparatus.

In order to achieve an as versatile as possible control of the MR apparatus it is arranged that the measuring method is given the directions of the field strength gradients of all gradient magnetic field devices of the MR apparatus which are used in the corresponding image, that is, in direction indications related to the co-ordinate system of the object to be examined and each time prior to the measurement. All imaging possibilities in the sense of the invention are thus utilized.

For further simplification of the operation it is arranged in accordance with the invention that the user receives on the control console each time a direction indication, related to the co-ordinate system of the object to be examined, for the direction of the field strength gradient of the gradient magnetic field device. The proposal made to the user is determined by the control algorithm of the MR apparatus, notably from the indications of the scan technique, the slice direction, and the direction of the phase encoding.

In order to ensure that the indications from the operator in respect of the shifting of the regions with deviantly resonating spins are also correctly interpreted by the method, it is advantageous when the axes of the co-ordinate system of the object to be examined extend parallel to the axes of the magnet co-ordinate system. This convention also holds when the object to be examined is arranged in the MR apparatus with preferred directions which do not extend parallel to the axes of the magnet co-ordinate system.

The invention also relates to a magnetic resonance imaging system comprising
a receiver system to acquire magnetic resonance signals are acquired from an object, the object to be examined having several main axes, and
a gradient system to apply several encoding gradients to spatially encode the magnetic resonance signals wherein
the gradient system is arranged to select the mutual orientation of the encoding gradients on the basis of the mutual orientation of the main axes of the object.

Because in the magnetic resonance imaging system of the invention the gradient system enables selection of the mutual orientation of the encoding gradients relative to the main axes of the object to be examined, shift of imaged fat containing tissue or shifts in the image caused by inhomogeneities are more easily controlled. Further, the invention relates to a computer program as defined in claim 11. The computer program of the invention when installed in the working memory of a processor of the magnetic resonance imaging system enables the magnetic resonance imaging system to perform the method of the invention. The computer program of the invention can be provided on a data carrier such as a CD-rom, or can be downloaded from a data network such as the world-wide web.

The invention will be described in detail hereinafter with reference to a special embodiment and drawings. Therein:

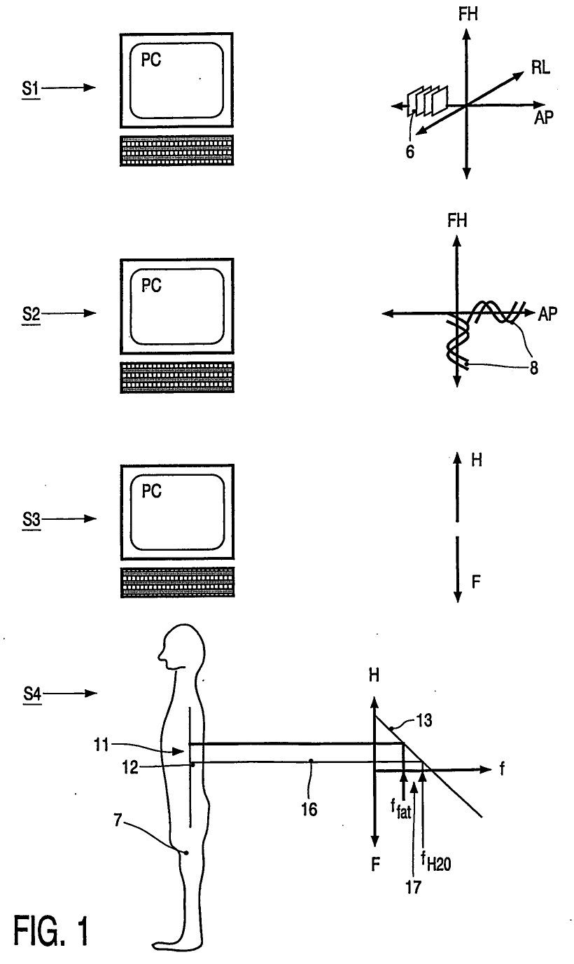

FIG. 1 is a diagrammatic representation of the execution of the method in accordance with the invention in the case of an echo planar sequence.

In FIG. 1 the individual steps of the method are denoted by the numbers 1 to 4 in conformity with their order of execution. The invention will be described on the basis of an example of an echo planar sequence.

In a first step 1 of the method, the operator selects the orientation in space of the slice 6 on a control console (PC). The operator of the MR apparatus then has the three spatial axes available (in this case right-left, anterior-posterior, feet-head; abbreviated as: RL, AP, FH). In conformity with the selected axis of the slice 6, a given slice of an object 7 to be examined is excited in a step of the measurement in that a magnetic field having a given field strength gradient 13 in the direction of the spatial axis selected for the slice 6 is applied across the object 7 to be examined and individual slices are selectively excited by means of a narrow-band RF pulse in conformity with the Larmor frequency of the slice to be excited. The user in this case selects a sagittal slice 6 (axis RL) from the three possibilities offered.

In a second step 2 of the method in accordance with the invention, the axial direction (FH, RL, AP) is defined in which position encoding 8 is to be performed by means of the phase of the proton precession. The axial direction FH of the position encoding 8 preferably lies in the slice plane of the excited slice. The operator can now choose from two spatial directions (FH, AP). The position encoding 8 is performed by application of a magnetic field having a given field strength gradient 13 in the direction of the phase encoding. In this case the axial direction FH, extending in the FH direction (feet-head) is selected.

In a third step 3 of the method in accordance with the invention the operator defines the orientation of the field strength gradient 13 along the axial direction FH determined in the second step. The control algorithm of the method in accordance with the invention offers the operator two possibilities (parallel or anti-parallel along the axis defined in the step 2) for the desired fat shift; the operator can choose freely among these two possibilities. In this case the direction H on the FH axis is chosen, implying an anti-parallel gradient direction along the FH axis, that is, in the direction F.

In a fourth step 4 the actual measurement is performed on the object 7 to be examined. A part which contains fat in the position 12 of the object 7 to be examined resonates in a manner which deviates from that of the part containing water in the position 12 of the object to be examined, that is, with a frequency which is approximately 3.5 ppm lower (frequency offset 17) than the parts containing water in the position 12 of the object 7 to be examined. The signal 16 received from the fat-containing part in the position 12 is interpreted by the analysis unit of the MR apparatus in a manner as if the received signal 16 has been emitted by a position situated further in the negative direction of the applied field strength gradient 13 (shift 11). The user has controlled the direction of the shift 11 of the fat-containing part in the position 12 of the object 7 to be examined via the selection of the orientation of the field strength gradient 13 in the third step 3 of the method in accordance with the invention.

1. A magnetic resonance imaging method in which
magnetic resonance signals are acquired from an object
wherein
several encoding gradients are applied to spatially encode the magnetic resonance signals and
the object to be examined has several main axes and
the mutual orientation of the encoding gradients is selected on the basis of the mutual orientation of the main axes of the object.

2. An method for the MR imaging of an object to be examined by means of an MR apparatus which includes at least one main magnetic field device and at least one gradient magnetic field device for position encoding,
In which a control algorithm controls measuring sequences in conformity with predetermined measuring parameters,
in which the magnetic moment of the spins is aligned on average in a preferred direction by means of a main magnetic field,
in which at least one gradient magnetic field which traverses at least partly the object to be examined and whose field strength exhibits a field strength gradient which is at least partly essentially constant in at least one spatial direction is applied for a given period of time,
in which the preferred direction of the magnetic field generated by means of the main magnetic field device defines an axis of a stationary magnetic co-ordinate system,
in which at least one further axis of the magnet co-ordinate system is defined by the direction of the field strength gradient of the magnetic field which can be generated by means of a gradient magnetic field device,
in which axes of a measuring co-ordinate system are defined each time by the direction of the slices and/or the direction of the phase encoding and/or the reading direction on the object to be examined, and
in which a co-ordinate system is associated with the object to be examined, wherein
the orientation of at least one field strength gradient, generated during the measurement, of a gradient magnetic field along an axis of the measuring co-ordinate system is provided as a measuring parameter for the method.

3. A method as claimed in claim 2, wherein the orientation of the field strength gradient of the gradient magnetic field is provided in the form of direction indications related to the co-ordinate system of the object to be examined.

4. A method as claimed in claim 2, wherein the control algorithm asks the operator of the MR apparatus for the desired direction of the shift of fat tissue in the image when the desired direction of the field strength gradient is selected on the control console.

5. A method as claimed in claim 4, wherein the desired direction of the field strength gradient is selected on the control console, the control algorithm asks the operator of the MR apparatus for the desired direction of the shift of fat tissue in the image along a predetermined axis of the co-ordinate system of the object to be examined, so that the operator has to choose among two possibilities only.

6. A method as claimed in claim 4, wherein the control algorithm determines the relevant axes for the shift of the fat tissue in the image from the other parameters of the position encoding and requests from the operator as input the respective desired direction of the shift of fat tissue in the image associated with these axes.

7. A method as claimed in claim 1, wherein the measuring method is given only the direction of the field strength gradient of a gradient magnetic field device in direction indications related to the co-ordinate system of the object to be examined, that is, each time prior to the measurement.

8. A method as claimed in claim 1, wherein the measuring method is given the directions of the field strength gradients of all gradient magnetic field devices of the MR apparatus which are used in the corresponding image, that is, in direction indications related to the co-ordinate system of the object to be examined and each time prior to the measurement.

9. A method as claimed in claim 1, wherein the user receives on the control console each time a direction indication, related to the co-ordinate system of the object to be examined, for the direction of the field strength gradient of the gradient magnetic field device.

10. A method as claimed in claim 1, wherein the axes of the co-ordinate system of the object to be examined extend parallel to the axes of the magnet co-ordinate system.

11. A computer program comprising instructions to select the mutual orientation of encoding gradients on the basis of the mutual orientation of the main axes of an object to be examined.

12. A magnetic resonance imaging system comprising
a receiver system to acquire magnetic resonance signals are acquired from an object, the object to be examined having several main axes, and
a gradient system to apply several encoding gradients to spatially encode the magnetic resonance signals wherein
the gradient system is arranged to select the mutual orientation of the encoding gradients on the basis of the mutual orientation of the main axes of the object.
